(19) United States
(12) Patent Application Publication
(10) Pub. No.: US 2013/0281688 A1
(43) Pub. Date: Oct. 24, 2013

(54) UNSATURATED FATTY ALCOHOL COMPOSITIONS AND DERIVATIVES FROM NATURAL OIL METATHESIS

(71) Applicants: Stephen A. Di Biase, River Forest, IL (US); Keith M. Wampler, Brookfield, IL (US); David R. Allen, Chicago, IL (US); Randal J. Bernhardt, Antioch, IL (US); Ryan Littich, Shorewood, IL (US)

(72) Inventors: Stephen A. Di Biase, River Forest, IL (US); Keith M. Wampler, Brookfield, IL (US); David R. Allen, Chicago, IL (US); Randal J. Bernhardt, Antioch, IL (US); Ryan Littich, Shorewood, IL (US)

(73) Assignee: Elevance Renewable Sciences, Inc, Woodridge, IL (US)

(21) Appl. No.: 13/867,542

(22) Filed: Apr. 22, 2013

Related U.S. Application Data
(60) Provisional application No. 61/637,574, filed on Apr. 24, 2012.
(60) Provisional application No. 61/780,490, filed on Mar. 13, 2013.

(51) Int. Cl. C07H 1/00; C07C 41/06; C07F 9/09; C07C 41/03; C07C 67/08; C07C 43/178; C07C 69/007

(52) U.S. Cl. CPC C07H 1/00; C07C 43/178; C07C 41/06; C07C 69/007; C07C 41/03; C07C 67/08; C07F 9/09
USPC 536/124; 568/873; 568/874; 568/680; 568/904; 554/223; 554/149; 536/1.11; 558/70; 558/114

(57) ABSTRACT

Unsaturated alcohol compositions are obtained by reducing a metathesis-derived hydrocarbyl unsaturated ester. Also disclosed is a process for preparing an unsaturated alcohol composition, where a metathesis derived hydrocarbyl carbonyl compound is reacted in the presence of a silane compound, an organic solvent, and a catalyst system prepared from a metallic complex and a reducing agent. This mixture is then hydrolyzed with a metallic base, and then mixed with organic solvent. The resultant mixture is then separated, washed, dried, and/or purified to produce the unsaturated alcohol composition. The unsaturated alcohol derivatives are useful in many end-use applications, including, for example, lubricants, functional fluids, fuels, functional additives for such lubricants, functional fluids and fuels, plasticizers, asphalt additives, friction reducing agents, plastics, and adhesives.

### CROSS REFERENCE TO RELATED APPLICATIONS

A claim of priority for this application under 35 U.S.C. §119(e) is hereby made to U.S. Provisional Patent Application No. 61/637,574, filed Apr. 24, 2012 and U.S. Provisional Patent Application No. 61/780,490, filed Mar. 13, 2013; the disclosures of which are incorporated herein by reference in their entireties.

### BACKGROUND

Fatty alcohol derivatives are used across a broad array of industries and end uses, including personal care, laundry and cleaning, emulsion polymerization, agricultural uses, oilfield applications, industrial compositions, and specialty foamers.

Fatty alcohols are usually made by reducing the corresponding fatty acids or esters, typically by catalytic hydrogenation. Often, the catalyst includes zinc or copper and chromium. U.S. Pat. No. 5,672,781, for instance, uses a CuCrO₄ catalyst to hydrogenate methyl esters from palm kernel oil, which has substantial unsaturation, to produce a mixture of fatty alcohols comprising about 52 wt. % of oleyl alcohol, a monounsaturated fatty alcohol. For additional examples, see U.S. Pat. Nos. 2,865,968; 3,193,586; 4,804,790; 6,683,224; and 7,169,959.

The fatty acids or esters used to make fatty alcohols and their derivatives are usually made by hydrolysis or transesterification of triglycerides, which are typically animal or vegetable fats. Consequently, the fatty portion of the acid or ester will typically have 6-22 carbons with a mixture of saturated and internally unsaturated chains. Depending on source, the fatty acid or ester often has a preponderance of C₁₆ to C₂₂ component. For instance, methanolysis of soybean oil provides the saturated methyl esters of palmitic (C₁₆) and stearic (C₁₈) acids and the unsaturated methyl esters of oleic (C₁₈ mono-unsaturated), linoleic (C₁₈ di-unsaturated), and α-linolenic (C₁₈ tri-unsaturated) acids. The unsaturation in these acids has either exclusively or predominantly cis-configuration.

Recent improvements in metathesis catalysts (see J.C. Mol, Green Chem. 4 (2002) 5) provide an opportunity to generate reduced chain length, monounsaturated feedstocks, which are valuable for making detergents and surfactants, from C₁₆ to C₂₂-rich natural oils such as soybean oil or palm oil. Soybean oil and palm oil can be more economical than, for example, coconut oil, which is a traditional starting material for making detergents. As Professor Mol explains, metathesis relies on conversion of olefins into new products by rupture and reformation of carbon-carbon double bonds mediated by transition metal carbene complexes. Self-metathesis of an unsaturated fatty ester can provide an equilibrium mixture of starting material, an internally unsaturated hydrocarbon, and an unsaturated diester. For instance, methyl oleate (methyl cis-9-octadecenoate) is partially converted to 9-octadecene and dimethyl 9-octadecene-1,18-dioate, with both products consisting predominantly of the trans-isomer. Metathesis effectively isomerizes the cis-double bond of methyl oleate to give an equilibrium mixture of cis- and trans-isomers in both the “unconverted” starting material and the metathesis products, with the trans-isomers predominating.

Cross-metathesis of unsaturated fatty esters with olefins generates new olefins and new unsaturated esters that can have reduced chain length and that may be difficult to make otherwise. For instance, cross-metathesis of methyl oleate and 3-hexene provides 3-dodecene and methyl 9-dodecenoate (see also U.S. Pat. No. 4,545,941). Terminal olefins are particularly desirable synthetic targets, and Elevance Renewable Sciences, Inc. recently described an improved way to prepare them by cross-metathesis of an internal olefin and an α-olefin in the presence of a ruthenium alkylidene catalyst (see U.S. Pat. Appl. Publ. No. 2010/0145086). A variety of cross-metathesis reactions involving an α-olefin and an unsaturated fatty ester (as the internal olefin source) are described. Thus, for example, reaction of soybean oil with propylene followed by hydrolysis gives, among other things, 1-decene, 2-undecenes, 9-decenoic acid, and 9-undecenoic acid. Despite the availability (from cross-metathesis of natural oils and olefins) of unsaturated fatty esters having reduced chain length and/or predominantly trans-configuration of the unsaturation, unsaturated fatty alcohols made from these feedstocks appear to be unknown.

In sum, traditional sources of fatty acids and esters used for making unsaturated fatty alcohols generally have predominantly (or exclusively) cis-isomers and lack relatively short-chain (e.g., C₁₀ or C₁₂) unsaturated fatty portions. Metathesis chemistry provides an opportunity to generate precursors having shorter chains and mostly trans-isomers, which could impart improved performance when the precursors are converted to downstream compositions.

### SUMMARY

In one aspect, the unsaturated alcohol compositions are obtained by reducing a metathesis-derived hydrocarbyl unsaturated ester. In another aspect, a process for preparing an unsaturated alcohol composition is disclosed where a metathesis derived hydrocarbyl carbonyl compound is reacted in the presence of a silane compound, an organic solvent, and a catalyst system prepared from a metallic complex and a reducing agent. This mixture is then hydrolyzed with a metallic base, and then mixed with organic solvent. The resultant mixture is then separated, washed, dried, and/or purified, as individual steps or in combinations thereof, to produce the unsaturated alcohol composition. Derivatives can be made by the polymerization of the metathesis-derived unsaturated alcohol with an individual or mixed alpha olefin stream. A sulfurized derivative can be made by reacting the metathesis-derived unsaturated alcohol with a sulfurizing reagent. An ester derivative can be made by reacting the metathesis-derived unsaturated alcohol with a carboxylic acid. An amine derivative can be made by reacting the metathesis-derived unsaturated alcohol with an amine compound. These metathesis-derived unsaturated alcohol derivatives are useful in many end-use applications, including, for example, lubricants, functional fluids, fuels, functional additives for such lubricants, functional fluids and fuels, plasticizers, asphalt additives, friction reducing agents, plastics, and adhesives.

### DETAILED DESCRIPTION

It is to be understood that unless specifically stated otherwise, references to “a,” “an,” and/or “the” may include one or more than one, and that reference to an item in the singular may also include the item in the plural.

In one aspect, the invention relates to derivatives made by one or more of unsaturated fatty alcohol compositions. In another aspect, the invention relates to fatty alcohol compositions which are made by reducing a metathesis-derived hydrocarbyl unsaturated ester. In another aspect of the invention, a process for preparing an unsaturated alcohol composition is disclosed where a metathesis derived hydrocarbyl carbonyl compound is reacted in the presence of a silane compound, an organic solvent, and a catalyst system prepared from a metallic complex and a reducing agent. This mixture is then hydrolyzed with a metallic base, and then mixed with organic solvent. The resultant mixture is then separated, washed, dried, and/or purified, as individual steps or in combinations thereof, to produce the unsaturated alcohol composition.

The hydrocarbyl unsaturated ester, preferably a C₅-C₃₅ unsaturated alkyl ester, and more preferably a C₁₀-C₁₇ unsaturated alkyl ester, used as a reactant is derived from metathesis of a natural oil. Preferably, the hydrocarbyl unsaturated esters are unsaturated alkyl esters. Traditionally, these materials, particularly the short-chain alkyl esters (e.g., methyl 9-decenoate or methyl 9-dodecenoate), have been difficult to obtain except in lab-scale quantities at considerable expense. However, because of the recent improvements in metathesis catalysts, these esters are now available in bulk at reasonable cost. Thus, the hydrocarbyl unsaturated esters are conveniently generated by self-metathesis of natural oils or cross-metathesis of natural oils with olefins, preferably α-olefins, and particularly ethylene, propylene, 1-butene, 1-hexene, 1-octene, and the like.

As used herein, the term “hydrocarbyl” or “hydrocarbyl group,” when referring to groups attached to the remainder of a molecule, refers to one or more groups having a purely hydrocarbon or predominantly hydrocarbon character. These groups may include: (1) purely hydrocarbon groups (i.e., aliphatic (alkyl), alicyclic, aromatic, branched, aliphatic- and alicyclic-substituted aromatic, aromatic-substituted aliphatic and alicyclic groups, as well as cyclic groups wherein the ring is completed through another portion of the molecule (that is, any two indicated substituents may together form an alicyclic group)); (2) substituted hydrocarbon groups (i.e, groups containing non-hydrocarbon substituents such as hydroxy, amino, nitro, cyano, alkoxy, acyl, halo, etc.); and (3) hetero groups (i.e., groups which contain atoms, such as N, O or S, in a chain or ring otherwise composed of carbon atoms). In general, no more than about three substituents or hetero atoms, or no more than one, may be present for each 10 carbon atoms in the hydrocarbyl group. The hydrocarbyl group may contain one, two, three, or four carbon-carbon double bonds.

Non-limiting examples of procedures for making hydrocarbyl unsaturated esters by metathesis are disclosed in WO 2008/048522, the contents of which are incorporated herein by reference. In particular, Examples 8 and 9 of WO 2008/048522 may be employed to produce methyl 9-decenoate and methyl 9-dodecenoate. Suitable procedures also appear in U.S. Pat. Appl. Publ. No. 2011/0113679, the teachings of which are incorporated herein by reference.

Preferably, at least a portion of the hydrocarbyl unsaturated ester has “Δ⁹” unsaturation, i.e., the carbon-carbon double bond in the ester is at the 9-position with respect to the ester carbonyl. In other words, there are preferably seven carbons between the ester carbonyl group and the olefin group at C9 and C10. For the C₁₁ to C₁₇ esters, an alkyl chain of 1 to 7 carbons, respectively is attached to C10. Preferably, the unsaturation is at least 1 mole % trans-Δ⁹, more preferably at least 25 mole % trans-Δ⁹, more preferably at least 50 mole % trans-Δ⁹, and even more preferably at least 80% trans-Δ⁹. The unsaturation may be greater than 90 mole %, greater than 95 mole %, or even 100% trans-Δ⁹. In contrast, naturally sourced fatty esters that have Δ⁹ unsaturation, e.g., methyl oleate, usually have ˜100% cis-isomers.

Although a high proportion of trans-geometry (particularly trans-Δ⁹ geometry) may be desirable in the metathesis-derived unsaturated fatty alcohol derivatives of the invention, the skilled person will recognize that the configuration and the exact location of the carbon-carbon double bond will depend on reaction conditions, catalyst selection, and other factors. Metathesis reactions are commonly accompanied by isomerization, which may or may not be desirable. See, for example, G. Djigoué and M. Meier, Appl. Catal., A 346 (2009) 158, especially FIG. 3. Thus, the skilled person might modify the reaction conditions to control the degree of isomerization or alter the proportion of cis- and trans-isomers generated. For instance, heating a metathesis product in the presence of an inactivated metathesis catalyst might allow the skilled person to induce double bond migration to give a lower proportion of product having trans-Δ⁹ geometry.

An elevated proportion of trans-isomer content (relative to the usual all-cis configuration of the naturally derived hydrocarbyl unsaturated ester) imparts different physical properties to unsaturated fatty alcohol derivatives, including, for example, modified physical form, melting range, compactability, and other important properties. These differences should allow formulators that use unsaturated fatty alcohol derivatives greater latitude or expanded choice as they use them in cleaners, detergents, personal care, agricultural uses, specialty foams, and other end uses.

Unsaturation can also impart advantages not seen in the corresponding saturated fatty alcohol derivatives. Because crystallinity is disrupted by the presence of a carbon-carbon double bond, unsaturated alcohol derivatives can sometimes be concentrated and formulated at higher actives levels—sometimes much higher—than their saturated counterparts. Thus, the seemingly minor structural change to a monounsaturated product can enable shipment of more concentrated products, reduce or eliminate the need for special handling equipment, and/or ultimately provide substantial cost savings.

Suitable metathesis-derived hydrocarbyl unsaturated esters derive from carboxylic acids. Preferably, the esters derive from C₅-C₃₅ carboxylic acids, more preferably from C₁₀-C₁₇ carboxylic acids. Examples include esters derived from 9-decylenic acid (9-decenoic acid), 9-undecenoic acid, 9-dodecylenic acid (9-dodecenoic acid), 9-tridecenoic acid, 9-tetradecenoic acid, 9-pentadecenoic acid, 9-hexadecenoic acid, 9-heptadecenoic acid, and the like.

Usually, cross-metathesis or self-metathesis of the natural oil is followed by separation of an olefin stream from a modified oil stream, typically by stripping or distilling out the more volatile olefins. The modified oil stream is then reacted with a lower alcohol, typically methanol, to give glycerin and a mixture of alkyl esters. This mixture normally includes saturated C₆-C₂₂ alkyl esters, predominantly C₁₆-C₁₈ alkyl esters, which are essentially spectators in the metathesis reaction. The rest of the product mixture depends on whether cross- or self-metathesis is used. When the natural oil is cross-metathesized with an α-olefin and the product mixture is transesterified, the resulting hydrocarbyl unsaturated ester mixture includes a C₁₀ unsaturated alkyl ester and one or more C₁₁ to C₁₇ unsaturated alkyl ester coproducts in addition to the glycerin by-product. The terminally unsaturated C₁₀ product is accompanied by different coproducts depending upon which α-olefin(s) is used as the cross-metathesis reactant. Thus, 1-butene gives a C₁₂ unsaturated alkyl ester, 1-hexene gives a C₁₄ unsaturated alkyl ester, and so on. The unsaturated alkyl esters are readily separated from each other and easily purified by fractional distillation. These hydrocarbyl unsaturated esters, preferably alkyl esters are excellent starting materials for making the inventive unsaturated alcohol derivative compositions.

Natural oils suitable for use as a feedstock to generate the hydrocarbyl unsaturated esters from self-metathesis or cross-metathesis with olefins are well known. Suitable natural oils include vegetable oils, algal oils, animal fats, tall oils, derivatives of the oils, and combinations thereof. Thus, suitable natural oils include, for example, soybean oil, palm oil, rapeseed oil, coconut oil, palm kernel oil, sunflower oil, safflower oil, sesame oil, corn oil, olive oil, peanut oil, cottonseed oil, canola oil, castor oil, linseed oil, tung oil, jatropha oil, mustard oil, pennycress oil, camellina oil, coriander oil, almond oil, wheat germ oil, bone oil, tallow, lard, poultry fat, fish oil, and the like. Soybean oil, palm oil, rapeseed oil, and mixtures thereof are preferred natural oils.

Genetically modified oils, e.g., high-oleate soybean oil or genetically modified algal oil, can also be used. Preferred natural oils have substantial unsaturation, as this provides a reaction site for the metathesis process for generating olefins. Particularly preferred are natural oils that have a high content of unsaturated fatty groups derived from oleic acid. Thus, particularly preferred natural oils include soybean oil, palm oil, algal oil, canola oil, and rapeseed oil.

A modified natural oil, such as a partially hydrogenated vegetable oil or an oil modified by a fermentation process, can be used instead of or in combination with the natural oil. When a natural oil is partially hydrogenated or modified by fermentation, the site of unsaturation can migrate to a variety of positions on the hydrocarbon backbone of the fatty ester moiety. Because of this tendency, when the modified natural oil is self-metathesized or is cross-metathesized with the olefin, the reaction products will have a different and generally broader distribution compared with the product mixture generated from an unmodified natural oil. However, the products generated from the modified natural oil are similarly converted to inventive unsaturated alcohol derivative compositions. In certain embodiments, the naturally occurring oil may be refined, bleached, and/or deodorized.

The other reactant in the cross-metathesis reaction is an olefin. Suitable olefins are internal or α-olefins having one or more carbon-carbon double bonds, and having between about 2 to about 30 carbon atoms. Mixtures of olefins can be used. Preferably, the olefin is a monounsaturated C₂-C₁₀ α-olefin, more preferably a monounsaturated C₂-C₈ α-olefin. Preferred olefins also include C₄-C₉ internal olefins. Thus, suitable olefins for use include, for example, ethylene, propylene, 1-butene, cis- and trans-2-butene, 1-pentene, isohexylene, 1-hexene, 3-hexene, 1-heptene, 1-octene, 1-nonene, 1-decene, and the like, and mixtures thereof.

Cross-metathesis is accomplished by reacting the natural oil and the olefin in the presence of a homogeneous or heterogeneous metathesis catalyst. The olefin is omitted when the natural oil is self-metathesized, but the same catalyst types are generally used. Suitable homogeneous metathesis catalysts include combinations of a transition metal halide or oxo-halide (e.g., WOCl₄ or WCl₆) with an alkylating cocatalyst (e.g., Me₄Sn). Preferred homogeneous catalysts are well-defined alkylidene (or carbene) complexes of transition metals, particularly Ru, Mo, or W. These include first and second-generation Grubbs catalysts, Grubbs-Hoveyda catalysts, and the like. Suitable alkylidene catalysts have the general structure:

M[X¹X²L¹L²(L³)ₙ]=Cₘ═C(R¹)R²

where M is a Group 8 transition metal, L¹, L², and L³ are neutral electron donor ligands, n is 0 (such that L³ may not be present) or 1, m is 0, 1, or 2, X¹ and X² are anionic ligands, and R¹ and R² are independently selected from H, hydrocarbyl, substituted hydrocarbyl, heteroatom-containing hydrocarbyl, substituted heteroatom-containing hydrocarbyl, and functional groups. Any two or more of X¹, X², L¹, L², L³, R¹ and R² can form a cyclic group and any one of those groups can be attached to a support.

First-generation Grubbs catalysts fall into this category where m=n=0 and particular selections are made for n, X¹, X², L¹, L², L³, R¹ and R² as described in U.S.

Pat. Appl. Publ. No. 2010/0145086 (“the '086 publication”), the teachings of which related to all metathesis catalysts are incorporated herein by reference.

Second-generation Grubbs catalysts also have the general formula described above, but L¹ is a carbene ligand where the carbene carbon is flanked by N, O, S, or P atoms, preferably by two N atoms. Usually, the carbene ligand is part of a cyclic group. Examples of suitable second-generation Grubbs catalysts also appear in the ‘086 publication.

In another class of suitable alkylidene catalysts, L¹ is a strongly coordinating neutral electron donor as in first- and second-generation Grubbs catalysts, and L² and L³ are weakly coordinating neutral electron donor ligands in the form of optionally substituted heterocyclic groups. Thus, L² and L³ are pyridine, pyrimidine, pyrrole, quinoline, thiophene, or the like.

In yet another class of suitable alkylidene catalysts, a pair of substituents is used to form a bi- or tridentate ligand, such as a biphosphine, dialkoxide, or alkyldiketonate. Grubbs-Hoveyda catalysts are a subset of this type of catalyst in which L² and R² are linked. Typically, a neutral oxygen or nitrogen coordinates to the metal while also being bonded to a carbon that is α-, β-, or γ- with respect to the carbene carbon to provide the bidentate ligand. Examples of suitable Grubbs-Hoveyda catalysts appear in the '086 publication.

The structures below provide just a few illustrations of suitable catalysts that may be used:

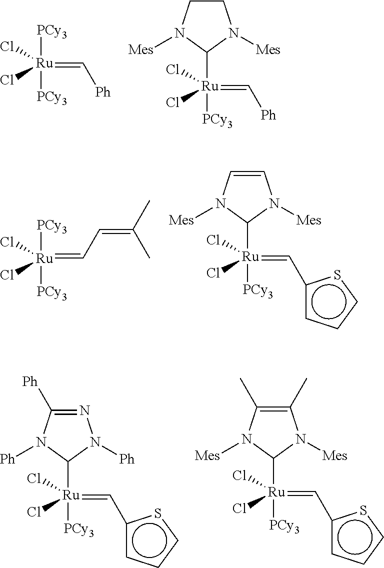

Heterogeneous catalysts suitable for use in the self- or cross-metathesis reaction include certain rhenium and molybdenum compounds as described, e.g., by J.C. Mol in Green Chem. 4 (2002) 5 at pp. 11-12. Particular examples are catalyst systems that include Re₂O₇ on alumina promoted by an alkylating cocatalyst such as a tetraalkyl tin lead, germanium, or silicon compound. Others include MoCl₃ or MoCl₅ on silica activated by tetraalkyltins.

For additional examples of suitable catalysts for self- or cross-metathesis, see U.S. Pat. No. 4,545,941, the teachings of which are incorporated herein by reference, and references cited therein. See also J. Org. Chem. 46 (1981) 1821; J. Catal. 30 (1973) 118; Appl. Catal. 70 (1991) 295; Organometallics 13 (1994) 635; Olefin Metathesis and Metathesis Polymerization by Ivin and Mol (1997), and Chem. & Eng. News 80(51), Dec. 23, 2002, p. 29, which also disclose useful metathesis catalysts. Illustrative examples of suitable catalysts include ruthenium and osmium carbene catalysts as disclosed in U.S. Pat. Nos. 5,312,940, 5,342,909, 5,710,298, 5,728,785, 5,728,917, 5,750,815, 5,831,108, 5,922,863, 6,306,988, 6,414,097, 6,696,597, 6,794,534, 7,102,047, 7,378,528, and U.S. Pat. Appl. Publ. No. 2009/0264672 A1, and PCT/US2008/009635, pp. 18-47, all of which are incorporated herein by reference. A number of metathesis catalysts that may be advantageously employed in metathesis reactions are manufactured and sold by Materia, Inc. (Pasadena, Calif.).

The unsaturated fatty alcohols (also referred to hereinbelow as simply “unsaturated alcohols”) are made by reacting a metathesis-derived hydrocarbyl unsaturated ester, preferably a C₅-C₃₅ unsaturated alkyl ester, and more preferably a C₁₀-C₁₇ unsaturated alkyl ester, with a reducing agent. As used herein, “unsaturated alcohols” typically have a hydrocarbyl chain length of between 6 and 24 carbon atoms. In some embodiments, the unsaturated alcohols have the general structure of R—CH═CH—(CH₂)₇—CH₂OH, wherein R is H or C₂-C₇ alkyl. In some embodiments, the fatty alcohol may be an unsaturated alcohol such as 9-decen-1-ol or 9-dodecen-1-ol.

Reduction of metathesis-derived hydrocarbyl unsaturated esters, preferably unsaturated alkyl esters, to produce the unsaturated alcohols is performed using well-known catalysts and procedures. The reducing agent is typically either a hydride reducing agent (sodium borohydride, lithium aluminum hydride, or the like) or molecular hydrogen in combination with a metal catalyst, frequently copper and/or zinc in combination with chromium, or a silane compound in combination with a metallic complex catalyst (see, e.g., U.S. Pat. Nos. 2,865,968; 3,193,586; 4,804,790; 5,124,491; 5,672,781; 5,831,133, 6,683,224; 7,169,959 and 7,208,643, and Mimoun, H. J., J. Org. Chem. 1999, 64, 2582-2589) the teachings of which are incorporated herein by reference).

The skilled person will appreciate that the reduction process, particularly when transition metal catalysts are used to convert the hydrocarbyl unsaturated esters to alcohols, can induce some degree of isomerization or migration of the carbon-carbon double bond from its original position. Moreover, because ester hydrogenation catalysts are not always completely selective, a minor proportion of the carbon-carbon double bonds, typically 10% or less, might be hydrogenated during the ester reduction, resulting in a mixed product that may have up to 10% of saturated fatty alcohols in addition to the desired unsaturated fatty alcohols.

In some embodiments, the process to prepare the unsaturated alcohols of the present invention is characterized in that a carbonyl compound, in particular, a hydrocarbyl unsaturated ester, is reacted with stoichiometric amounts of a silane compound, in the presence of a catalyst system prepared from a metallic complex and a reducing agent. Preferably, the unsaturated alcohols comprise 9-decen-1-ol or 9-dodecen-1-ol, and the hydrocarbyl unsaturated ester comprises methyl-9-decenoate or methyl-9-dodecenoate. The silane compound can be selected from the group consisting of alkyltrihydrosilanes, aryltrihydrosilanes, dialkyldihydrosilanes, diaryldihydrosilanes, trialkylhydrosilanes, triarylhydrosilanes, alkylhydrosiloxanes, arylhydrosiloxanes, polyalkylhydrosiloxanes and the like, individually or in combinations thereof. Preferably, the silane compound is polymethylhydrosiloxane. The catalyst system can be obtained in situ, in the reaction medium or be prepared separately, and comprises a metallic complex of general formula MXₙ, wherein M represents a transition metal selected from the group consisting of zinc, cadmium, manganese, cobalt, iron, copper, nickel, ruthenium and palladium, X an anion comprising a halide, a carboxylate or any anionic ligand, wherein X is selected from the group consisting of chloride, bromide, iodide, carbonate, isocyanate, cyanide, phosphate, acetate, propionate, 2-ethylhexanoate, stearate or naphthenate of one of the above-mentioned metals, individually or in combinations thereof, and n is a number comprised between 1 and 4. In some embodiments, X will be reacted with a reducing agent selected from the group consisting of a hydride, wherein the hydride can be an alkaline hydride such as lithium, sodium or potassium hydride, or an alkaline earth hydride such as magnesium or calcium hydride, or a boron hydride such as BH₃, a metallic borohydride MBH₄ (M=Li, Na, K) or M(BH₄)₂ (M=Mg, Zn, Ca), an alkylborane Rₙ BH(4-n) M (R=alkyl, n=1 to 3, M=alkaline metal), a(RO)ₙ BH(4-n) M (R=alkyl, n=1 to 3, M=alkaline metal), or an aluminum hydride AlH₃, AlHₙ R₃-n (R=alkyl), MAlH₄ (M=Li, Na, K), MAN, (OR)₄-n (M=Li, Na, K), or an organic magnesium compound of formula RMgX (R=alkyl, X═Cl, Br, I), or an organic lithium compound RLi (R=alkyl, for example C₁ to C₄ or aryl), individually or in combinations thereof, in order to generate the active catalyst according to the invention. Preferably, M is zinc, X is a carboxylate such as 2-ethylhexanoate, n is 2, and the reducing agent is sodium borohydride, thus providing for a zinc 2-ethylhexanoate complex.

In some embodiments, the metallic complex and reducing agent, either individually, or in combination thereof, may be mixed with an inert organic solvent, for example, an ether such as methyltertbutylether, diisopropylether, dioxane, tetrahydrofuran, ethyleneglycol dimethylether, or an aliphatic hydrocarbon such as heptane, petroleum ether, octane, cyclohexane, or aromatic as benzene, toluene, xylene or mesitylene, individually or in combinations thereof. Preferably, the solvent is diisopropylether.

When the catalyst system is prepared in situ, the chosen metallic complex, (preferably zinc 2-ethylhexanoate) will be reacted with the reducing agent (preferably sodium borohydride) in an appropriate organic solvent (preferably diisopropyl ether) with the carbonyl compound (preferably an unsaturated hydrocarbyl ester such as methyl-9-decenoate or methyl-9-dodecenoate) at room temperature. After full release of the formed hydrogen, the carbonyl compound to be reduced will be introduced and heated, and thereafter the silane compound (preferably polymethylhydrosiloxane) is added into the solution. The typical consumption of PMHS will be about 2.2 equivalents for the reduction of esters. The resulting solution is hydrolyzed by reacting the solution with an aqueous or alcoholic solution of a metallic base, such as sodium hydroxide, potassium hydroxide, calcium oxide or sodium carbonate (preferably potassium hydroxide), individually or in combinations thereof, and then adding an appropriate organic solvent. Once the hydrolysis is complete, formation of two phases is generally observed, with the desired alcohol being in the organic phase. This organic phase is then separated, washed, dried, and /or purified, as individual steps or in combination thereof, to produce the unsaturated alcohol.

In some embodiments, the unsaturated alcohol can be produced by the selective hydrogenation of methyl oleate (methyl-9-octadecenoate) into oleyl alcohol (methyl-9-octadecen-1-ol). This hydrogenation can be carried out over bimetallic catalysts containing cobalt and tin, or ruthenium and tin. Other methods to produce unsaturated alcohols are provided in U.S. Pat. Nos. 5364986 and 6229056, the teachings of which are incorporated herein by reference.

Suitable hydrocarbyl unsaturated esters can be generated by transesterifying a metathesis-derived triglyceride. For example, cross-metathesis of a natural oil with an olefin, followed by removal of unsaturated hydrocarbon metathesis products by stripping, and then transesterification of the modified oil component with a lower alkanol under basic conditions provides a mixture of hydrocarbyl unsaturated esters, preferably unsaturated alkyl esters. The hydrocarbyl unsaturated ester mixture can be purified to isolate particular alkyl esters prior to making the unsaturated alcohols and inventive derivatives.

As used herein, “derivatives” includes not only chemical compositions or materials resulting from the reaction of unsaturated fatty alcohol(s) with at least one other reactant to form a reaction product, and further downstream reaction products of those reaction products as well, but does not include chemical compositions or materials that result from the reaction of unsaturated fatty alcohols with at least one alkoxylating, sulfating, sulfonating, or sulfitating agent.

For example, the unsaturated alcohol may be further reacted into one or more alcohol derivatives, wherein such alcohol derivatives may be generated by dehydration of an alcohol to form alkenes, oxidation of an alcohol to form aldehydes or ketones, substitution of an alcohol to form alkyl halides, and esterification. Such alcohol derivatives can have a very large variety of structures and include linear-, branched- or cyclic-aliphatic monoalcohol derivatives, diol derivatives and/or polyol derivatives; and aromatic or heterocyclic alcohol derivatives including natural alcohol derivatives, e.g., sugars and/or heteroatom-functional aliphatic alcohol derivatives such as aminoalcohol derivatives. In general, alcohol derivatives can be saturated or unsaturated, linear or have branches of a great variety of types known in the art depending on the size and position of branching moieties or, in other terms, analytical characterization (e.g., by NMR), performance properties, or the process by which the alcohol derivatives are made.

For example, saccharide-derived fatty alcohol compositions readily derived from the disclosed unsaturated alcohols include alkyl polyglucosides. These all-natural alkyl polyglucosides can serve as nonionic surfactants, and are prepared by acid-catalyzed direct glycosidation of unsaturated fatty alcohols, or transglycosidation of lower polyglucosides (e.g., butyl polyglucoside) with unsaturated alcohols. Anionic derivatives may also be prepared from alkyl polyglucosides by sulfation (e.g., with chlorosulfonic acid, oleum, sulfur trioxide, etc.), phosphorylation (e.g., with dibenzyl diisopropylamino phosphoramidite), esterification (e.g., with maleic anhydride, citric acid) and subsequent sulfation/phosphorylation, and glucose C₆-alcohol selective oxidation to the corresponding carboxylate.

Other accessible saccharide-based unsaturated fatty alcohol compositions are alkyl glyceryl ethers. Alkyl glyceryl ethers are prepared by the alkylation of unsaturated fatty alcohols with glycidol, in the presence of an acidic or alkaline catalyst. The hydrophile-lipophile balance of this class of nonionic surfactant is readily modified by the number of glycidol moieties added to the fatty alcohol substrate. Alkyl glyceryl ether derivatives may be further transformed into anionic surfactants by sulfation using any of the conventional reagents (chlorosulfonic acid, oleum, sulfur trioxide, etc.).

Additional amphiphilic derivatives are accessible from the unsaturated alcohols disclosed herein. A number of anionic surfactants may be prepared, including di-basic sulfosuccinate half esters and mono-basic sulfosuccinate diesters. These mono- and diesters are derived from maleic anhydride by ring-opening esterification with fatty alcohols then Michael addition of aqueous sodium bisulfite to the intermediate maleic acid mono- or diester.

The synthesis of novel phosphoric acid mono-, di-, and tri-esters is also accessible from the unsaturated fatty alcohols. Mono- and dibasic acid esters of phosphoric acid may be prepared by phosphorylation (“phosphation”) of fatty alcohols using phosphorus pentoxide. Owing to the presence of polyphosphoric acid and o-phosphoric acid in phosphorus pentoxide, molar ratios of mono- to diester of ˜1.2:1 are generally observed. Triesters of phosphoric acid, conversely, are most readily prepared by esterification using phosphorus oxychloride in the presence of a tertiary amine as HCl scavenger.

The preparation of other anionic surfactant species is supported by oxyalkylenation of the unsaturated fatty alcohols. Carboxymethylation of fatty alcohol alkoxylates, either by alkylation of the terminal alcohol using sodium chloroacetate or by catalytic alkaline oxidation of the terminal alcohol to the corresponding acid, provides alkyl ether carboxylates. Carboxyethylation of fatty alcohol alkoxylates, either by cyanoethylation and subsequent hydrolysis or by alkylation using sodium β-chloropropionate, also generates alkyl ether carboxylates (one carbon homologue of those derived from chloroacetate). In some embodiments, oxyalkylenated unsaturated fatty alcohols (e.g., fatty alcohol ethoxylates, fatty alcohol propoxylates) may be used as substrates in the preparation of the above alkenyl sulfosuccinates, alkenyl phosphates, alkenyl glyceryl ethers.

In some embodiments, certain derivatives such as polymerized materials can be generated by reacting an individual or mixed alpha olefins stream with unsaturated alcohols, preferably metathesis-derived unsaturated alcohols. Such polymerized materials can be useful as synthetic base stocks for preparing lubricants or functional fluids, wherein such synthetic base stocks provide good solvency and lubricity while being miscible with conventional hydrocarbon lubricants. Such polymerized materials can also be useful as an additive that can be incorporated with a base stock to create a finished lubricant.

In particular, 9-decen-1-ol derived from methyl-9-decenoate, with individual or mixed alpha olefins such as 1-decene and/or 1-dodecene can be polymerized to make synthetic base stocks for preparing lubricants or functional fluids, or as an additive in a finished lubricant. The polymerization can be carried out using conventional polymerization techniques. The polymerization may comprise a batch process, a continuous process, or a staged process. Polymerization may be effected either via the one or more carbon-carbon double bonds, the functional groups and/or the additional functionality provided by the reactants. The polymerization may involve employing one or more cationic, free radical, anionic, Ziegler-Natta, organometallic, metallocene, or ring-opening metathesis polymerization (ROMP) catalysts. Free radical initiators may include azo compounds, peroxides, light (photolysis), and combinations thereof. The azo compounds may include azobisisobutyronitrile (AIBN), 1,1′-azobis(cyclohexanecarbonitrile), and the like, and combinations thereof. The peroxide compounds may include benzoyl peroxide, methyl ethyl ketone peroxide, tert-butyl peroxide, di-tert-butylperoxide, t-butyl peroxy benzoate, di-t-amyl peroxide, lauroyl peroxide, dicumyl peroxide, tert-butyl perpivalate, di-tert-amyl peroxide, dicetyl peroxydicarbonate, tert -butyl peracetate, 2,2-bis(tert-butylperoxy)butane, 2,5-bis(tert-butylperoxy)-2,5-dimethyl-3-hexyne, 2,5-bis(tert-butylperoxy)-2,5-dimethylhexane, 2,5-dimethyl-2,5-di(t-butyl peroxy)hexane, and the like, and combinations thereof. The free radical initiator may comprise di-t-butyl peroxide. The anionic catalyst may include butyl lithium. Optionally, it may be desirable to control the molecular weight and molecular architecture prior to or during polymerization by the addition of a chain transfer agent. Suitable chain transfer agents may include dodecanethiol, t-nonylthiol, tetramethylsilane, cyclopropane, methane, t-butanol, ethane, ethylene oxide, 2,2-dimethylpropane, benzene, carbon tetrachloride, and bromotrichloromethane.

Polymerization may be achieved under cationic conditions and, in such embodiments, the acid catalyst may comprise a Lewis Acid, a Brønsted acid, or a combination thereof. The Lewis acids may include boron triflouride (BF₃), AlCl₃, zeolite, and the like, and complexes thereof, and combinations thereof. The Brønsted acids may include HF, HCl, phosphoric acid, acid clay, and the like, and combinations thereof. Polymerization may be achieved using a promoter (e.g., an alcohol) or a dual promoter (e.g., an alcohol and an ester) as described U.S. Pat. Nos. 7,592,497 B2 and 7,544,850 B2, the teachings of which are incorporated by reference.

The polymerization catalysts described herein may be supported on a support. For example, the catalysts may be deposited on, contacted with, vaporized with, bonded to, incorporated within, adsorbed or absorbed in, or on, one or more supports or carriers. The catalysts described herein may be used individually or as mixtures. The polymerizations using multiple catalysts may be conducted by addition of the catalysts simultaneously or in a sequence.

In some embodiments, using catalytic amounts of Lewis acid catalyst such as boron triflouride, along with an alcohol promoter, can complex with boron triflouride to form a coordination compound which is catalytically active for the polymerization reaction. In some embodiments, 9-decen-1-ol or 9-dodecen-1-ol can serve as the alcohol promoter in the reaction, allowing for superior economics.

In addition to the above polymerization, the aforementioned unsaturated alcohols can be reacted with sulfurizing reagents, such as solid, particulate, or molten forms of elemental sulfur, sulfur halides, hydrogen sulfide, phosphorus sulfide, aromatic sulfide, alkyl sulfide, sulfurized olefin, sulfurized oil, sulfurized fatty ester, diester sulfide, or a mixture of two or more thereof. Such reaction can generate hydrocarbyl sulfur containing materials useful in specialty chemical applications including but not limited to lubricants or functional fluids, or as an additive in a finished lubricant, asphalt compositions, polymeric materials as property enhancing additives such as plasticizers and anti-oxidants.

Reactions of the alcohol moiety in an unsaturated alcohol may create new monomers useful in making high performance polymers and oligomers. Some non-limiting examples are reacting the alcohol moiety with carboxylic acids to make olefinic esters which can be polymerized using the methods mentioned above. These carboxylic acids may comprise one or more monobasic and/or polybasic unsaturated carboxylic acids. The monobasic carboxylic acids may comprise one or more compounds represented by the formula

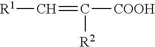

wherein R¹ and R² are independently hydrogen or hydrocarbyl groups. R¹ and R² independently may be hydrocarbyl groups containing 5 to about 35 carbon atoms, or from 1 to about 12 carbon atoms, or from 1 to about 4 carbon atoms.

The polybasic carboxylic acid may comprise one or more alpha, beta, or internally unsaturated dicarboxylic acids. These may include those wherein a carbon-carbon double bond is in an alpha, beta, or internal position to at least one of the carboxy functions, or in an alpha, beta, internal position to both of the carboxy functions. The carboxy functions of these compounds may be separated by up to about 4 carbon atoms, or about 2 carbon atoms. The olefinic esters may comprise a hydrocarbyl chain of from about 3 to about 35 carbon atoms, or from about 6 to about 24 carbon atoms, or from about 8 to about 18 carbon atoms, or about 10 to 12 carbon atoms, and 1, 2, 3 or 4 internal carbon-carbon double bonds.

The alcohol group can be further reacted with an amine to give an olefinic amine which may have unique properties when evaluated alone and in combination with adjuvants in the aforementioned applications. The amine may contain one or more primary and/or secondary amino groups, or be a mono-substituted amine, di-substituted amine, poly-substituted amine, or a mixture of two or more thereof. In some embodiments, the olefinic amine can have high value as a novel monomer for making lubricants or functional fluids or as an additive in a finished lubricant.

Polymers made from the unsaturated alcohol and amine can be further reacted with electrophiles to yield derivatives useful as plasticizers, lubricants lubricant additives and intermediates, antimicrobial, friction reducing agents, plastics, coatings, adhesives and other compositions.

Primary and secondary amines arising from the amination of unsaturated fatty alcohols may be further derivatized on treatment with acrylonitrile, in the presence of a suitable alkaline or acidic catalyst. The resulting mono- or di-cyanoethylated amines, products of Michael addition, are often reduced to provide the corresponding propylamines (e.g. polyamines). This process of Michael addition, and then reduction, can be performed iteratively to produce higher polyamines.

Primary, secondary, and tertiary amines derived from unsaturated fatty alcohols may be derivatized via salt formation, employing a wide variety of mineral and organic acids (e.g., acetic acid) in the production of such fatty amine salts. The resulting amine salts have utility in a variety of applications, for example, as dispersants and anti-caking agents in agrochemical, petrochemical and water remediation applications.

### General Note Regarding Chemical Structures:

As the skilled person will recognize, products made in accordance with the invention are typically mixtures of cis- and trans- isomers. Except as otherwise indicated, all of the structural representations provided herein show only a trans-isomer. The skilled person will understand that this convention is used for convenience only, and that a mixture of cis- and trans-isomers is understood unless the context dictates otherwise. Structures shown often refer to a principal product that may be accompanied by a lesser proportion of other components or positional isomers. Thus, the structures provided represent likely or predominant products.

Some specific examples of C₁₀, C₁₂, C₁₄, and C₁₆-based unsaturated alcohols used to make inventive derivatives appear below:

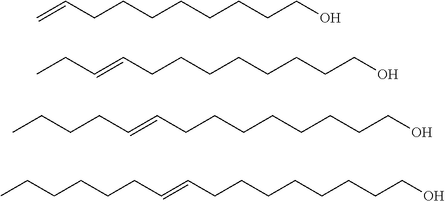

Some unsaturated fatty alcohol compositions used to make the inventive derivatives have the general structure:

R—C₉H₁₆—CH₂OH

wherein R is H or C₂-C₇ alkyl. Preferably, the fatty alcohol compositions have the general structure:

R—CH═CH—(CH₂)₇—CH₂OH

wherein R is H or C₂-C₇ alkyl.

The invention includes a process for making derivatives. The process comprises first reducing a metathesis-derived hydrocarbyl unsaturated ester, preferably a C₅-C₃₅ unsaturated alkyl ester, and more preferably a C₁₀-C₁₇ unsaturated alkyl ester, to produce an unsaturated fatty alcohol composition. The fatty alcohol composition is then converted to a derivative. Suitable reagents and processes for effecting the reduction have already been described.

A composition comprising at least one unsaturated fatty alcohol derivative is provided. The composition may be an aqueous system or provided in other forms. The unsaturated fatty alcohol derivatives described herein may be incorporated into various formulations and used as lubricants, functional fluids, fuels and fuel additives, additives for such lubricants, functional fluids and fuels, plasticizers, asphalt additives, friction reducing agents, antistatic agents in the textile and plastics industries, flotation agents, gelling agents, epoxy curing agents, corrosion inhibitors, pigment wetting agents, in cleaning compositions, plastics, coatings, adhesives, surfactants, emulsifiers, skin feel agents, film formers, rheological modifiers, solvents, release agents, conditioners, and dispersants, hydrotropes, etc. Where applicable, such formulations may be used in end-use applications including, but not limited to, personal care, as well as household and industrial and institutional cleaning products, oil field applications, gypsum foamers, coatings, adhesives and sealants, agricultural formulations, to name but a few. Thus, the unsaturated fatty alcohol derivatives described herein may be employed as or used in applications including, but not limited to bar soaps, bubble baths, shampoos, conditioners, body washes, facial cleansers, hand soaps/washes, shower gels, wipes, baby cleansing products, creams/lotions, hair treatment products, anti-perspirants/deodorants, enhanced oil recovery compositions, solvent products, gypsum products, gels, semi-solids, detergents, heavy duty liquid detergents (HDL), light duty liquid detergents (LDL), liquid detergent softener antistat formulations, dryer softeners, hard surface cleaners (HSC) for household, autodishes, rinse aids, laundry additives, carpet cleaners, softergents, single rinse fabric softeners, I&I laundry, oven cleaners, car washes, transportation cleaners, drain cleaners, defoamers, anti-foamers, foam boosters, anti-dust/dust repellants, industrial cleaners, institutional cleaners, janitorial cleaners, glass cleaners, graffiti removers, concrete cleaners, metal/machine parts cleaners, pesticide emulsifiers, agricultural formulations and food service cleaners.

The unsaturated alcohol derivatives may be incorporated into, for example, various compositions and used as lubricants, functional fluids, fuels, additives for such lubricants, functional fluids and fuels, plasticizers, asphalt additives and emulsifiers, friction reducing agents, plastics, coatings, adhesives, surfactants, emulsifiers, skin feel agents, film formers, rheological modifiers, biocides, biocide potentiators, solvents, release agents, conditioners, and dispersants, etc. Where applicable, such compositions may be used in end-use applications including, but not limited to, personal care liquid cleansing products, conditioning bars, oral care products, household cleaning products, including liquid and powdered laundry detergents, liquid and sheet fabric softeners, hard and soft surface cleaners, sanitizers and disinfectants, and industrial cleaning products, emulsion polymerization, including processes for the manufacture of latex and for use as surfactants as wetting agents, dispersants, solvents, and in agriculture applications as formulation inerts in pesticide applications or as adjuvants used in conjunction with the delivery of pesticides including agricultural crop protection turf and ornamental, home and garden, and professional applications, and institutional cleaning products. They may also be used in oil field applications, including oil and gas transport, production, stimulation and drilling chemicals and reservoir conformance and enhancement, organoclays for drilling muds, specialty foamers for foam control or dispersancy in the manufacturing process of gypsum, cement wall board, concrete additives and firefighting foams, paints and coatings and coalescing agents, paint thickeners, adhesives, or other applications requiring cold tolerance performance or winterization (e.g., applications requiring cold weather performance without the inclusion of additional volatile components).

The formulations mentioned above commonly contain, in addition to the unsaturated alcohol derivatives disclosed herein, one or more other components for various purposes, such as surfactants, anionic surfactants, cationic surfactants, ampholtyic surfactants, zwitterionic surfactants, mixtures of surfactants, builders and alkaline agents, enzymes, adjuvants, fatty acids, odor control agents and polymeric suds enhancers, and the like.

The following examples merely illustrate the invention. The skilled person will recognize many variations that are within the spirit of the invention and scope of the claims.

### EXAMPLES

### Reduction of Methyl 9-Decenoate to 9-Decen-1-ol (A10-1)

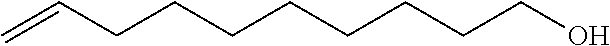

The procedure of Micovic and Mihailovic (J. Org. Chem. 18 (1953) 1190) is generally followed. Thus, a 5-L flask equipped with a mechanical stirrer, thermocouple, addition funnel, and nitrogen inlet is charged with tetrahydrofuran (“THF,” 3 L). The flask is immersed in an isopropanol/CO₂ bath. Lithium aluminum anhydride (LAH) pellets (133.8 g) are charged to the flask with stirring. Methyl 9-decenoate (250 g) is charged to the addition funnel and diluted with THF to the maximum capacity of the funnel (500 mL). The ester solution is added dropwise to the LAH suspension at a rate that maintains the reaction temperature below 20° C. The funnel is refilled with pure ester (750 g; total of 1000 g) due to the large volume of the reaction mixture, and the addition continues. Total addition time of the ester: 5 h. Once the addition is complete, the reaction temperature is ˜15° C. and stirring continues for 30 min. ¹H NMR analysis shows complete conversion of the ester to the desired alcohol.

Deionized water (135 g) is added slowly via the addition funnel while keeping the temperature below 20° C. Hydrogen evolution appears to cease after approximately half of the water is added. The viscosity of the mixture increases, but it remains stirrable. The flask is removed from the cooling bath, and aqueous sodium hydroxide (15% aq. NaOH, 135 g) is added. During this addition, the reaction mixture thickens and quickly becomes an unstirrable slurry that has to be broken up with a spatula. Addition of the remaining NaOH solution proceeds without incident. Following the 15% NaOH addition, deionized water (3×135 g) is added. The slurry stirs for 20 min. and then stands overnight at room temperature. The mixture is filtered through a Buchner funnel, and the filter cake is washed with additional THF (2×500 mL) and then acetone (2×500 mL). The filtrates are combined and concentrated. ¹H NMR analysis of the remaining oil reveals a clean alcohol product. The crude alcohol is transferred to a round-bottom flask and heated to 50° C. Full vacuum is slowly applied to remove low-boiling volatiles. The remaining crude product is then vacuum distilled, collecting the product that boils at 95-98° C. (97.5-100° C. pot temperature). Yield of A10-1: 834.7 g (98.3%). Purity (by GC analysis): 99.7%. Hydroxyl value: 355.5 mg KOH/g sample; iodine value: 162.2 g I₂/100 g sample. ¹H NMR (δ, CDCl₃): 5.8 (CH₂═CH—); 4.95 (CH₂═CH—); 3.6 (—CH₂—OH). The procedure is repeated four times using 1 kg of ester in each reduction.

### Reduction of Methyl 9-Dodecenoate to 9-Dodecen-1-ol (Al2-1)

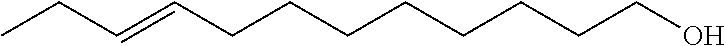

The procedure used to prepare A10-1 is generally followed using THF (3 L), lithium aluminum hydride pellets (116 g), and methyl 9-dodecenoate (1000 g total).

The usual work-up follows, first with deionized water (120 g), then aqueous sodium hydroxide (15% aq. NaOH, 120 g). Following the 15% NaOH addition, deionized water (360 g) is added. The slurry stirs for 20 min. and then stands overnight at room temperature. The mixture is filtered through a Buchner funnel, and the filter cake is washed with additional THF (4×1 L). The filtrates are combined and concentrated.

The procedure is repeated five times using 1 kg of methyl 9-dodecenoate for each run, and the crude alcohol products are combined and distilled as described above for the preparation of A10-1. Yield of Al2-1: 4262.8 g (98.2%). Purity (by GC analysis): 99.4%. Hydroxyl value: 302.8 mg KOH/g sample; iodine value: 133.2 g I₂/100 g sample. ¹H NMR (δ, CDCl₃): 5.4 (—CH═CH—); 3.6 (—CH₂—OH); 0.9 (CH₃—).

### Reductions of Methyl 9-Decenoate to 9-Decen-1-ol and Methyl 9-Dodecenoate to 9-Dodecen-1-ol using Polymethylhydrosiloxane (PMHS)

Materials. Methyl 9-decenoate (lot no. 184-133) and methyl 9-dodecenoate (lot no. 184-133) were obtained from Materia, Inc. (Pasadena, Calif.). Poly(methylhydrosiloxane) (Alfa-Aesar, Ward Hill, Mass.; lot no. 10111148), zinc bis(2-ethylhexanoate) (Strem Chemicals, Newburyport, Mass.; lot no. A4174040), sodium borohydride (Strem Chemicals, Newburyport, Mass.; lot no. 19957400), and diisopropylether (Acros Organics, N.J.; lot no. B0520262) were purchased from their respective suppliers.

Synthesis of 9-decen-1-ol. Zinc bis(2-ethylhexanoate) (328.0 mg, 1.085 mmol) was dissolved in 15 mL ⁱPr₂O (diisopropyl ether) and this solution was transferred into a 100 mL round bottom flask equipped with a magnetic stir bar. NaBH₄ (41.0 mg, 1.085 mmol) was added slowly to the rapidly stirring solution. Once gas evolution had ceased (<2 min), methyl 9-decenoate (10.00 g, 54.26 mmol) was added to the prepared catalyst solution and then the flask was fitted with a reflux condenser and placed in a silicone oil bath. The solution was then brought to reflux under air for three hours, after which time polymethylhydrosiloxane (PMHS) (7.765 g, 119.3 mmol) was added and the solution was refluxed for an additional three hours. The slightly turbid, colorless solution was then cooled to room temperature and slowly treated with a solution of 10 g KOH in 30 mL of water. The reaction proceeded first with hydrogen gas evolution, due to excess silane, and then the formation of a white precipitate. The mixture was transferred to a separatory funnel with an additional 15 mL of ⁱPr₂O and the bottom layer was removed. The upper layer was washed with 3×50 mL of brine. The organic layer was then dried over Na₂SO₄, which was subsequently removed by filtration through a medium porosity sintered glass frit. All volatiles were then removed to ˜500 mTorr to give 7.121 g (45.57 mmol, 84% yield) of a viscous, colorless oil. The product was identified by comparison of its mass spectrum (GCMS-EI) with the spectrum of 9-decen-1-ol in the NIST database. The IR spectrum of the pure oil (vide supra) was also consistent with that in the NIST spectral database.

Synthesis of 9-dodecen-1-ol. The procedure for the synthesis of 9-decen-1-ol detailed above was employed on 10.00 g methyl 9-dodecenoate (47.13 mmol) using 332 mg zinc bis(2-ethylhexanoate (0.9426 mmol) and 36 mg sodium borohydride (0.9426 mmol) as catalyst and 6.739 g poly(methylhydrosiloxane) (103.7 mmol) as reductant. The only deviation from the procedure above was that the quenching solution of 10 g KOH must be 50:50 MeOH:H₂O due to the higher lipophilicity of the substrate. Following the above mentioned work-up, 6.953 g of a viscous, colorless oil (37.75 mmol, 80% yield). The product was identified by comparison of its mass spectrum with the spectrum of 9-dodecen-1-ol in the NIST database. The IR spectrum of 9-dodecen-1-ol was not available in the NIST database but the spectrum obtained from the product was consistent with the formulation.

While the invention has been explained in relation to various embodiments and examples, it is to be understood that various modifications thereof will become apparent to those skilled in the art upon reading the specification. Therefore, it is to be understood that the invention disclosed herein includes any such modifications that may fall within the scope of the appended claims.

We claim:

1. An unsaturated alcohol composition made by reducing a metathesis-derived hydrocarbyl unsaturated ester.

2. The composition of claim 1, wherein the hydrocarbyl unsaturated ester comprises a C₅-C₃₅ unsaturated alkyl ester.

3. The composition of claim 1, wherein the hydrocarbyl unsaturated ester comprises methyl-9-decenoate.

4. The composition of claim 1, wherein the hydrocarbyl unsaturated ester comprises methyl-9-dodecenoate.

5. The composition of claim 1, having the general structure:
R—CH═CH—(CH₂)₇—CH₂OH
wherein R is H or C₂-C₇ alkyl.

6. The composition of claim 1 wherein the hydrocarbyl unsaturated ester is made by cross-metathesis of a natural oil with an olefin, followed by stripping to remove unsaturated hydrocarbons from a modified oil component, followed by transesterification of the modified oil component with an alkanol under basic conditions to generate the hydrocarbyl unsaturated ester.

7. The composition of claim 6, wherein the natural oil is selected from the group consisting of soybean oil, palm oil, rapeseed oil, coconut oil, palm kernel oil, sunflower oil, safflower oil, sesame oil, corn oil, olive oil, peanut oil, cottonseed oil, canola oil, castor oil, linseed oil, tung oil, jatropha oil, mustard oil, pennycress oil, camellina oil, coriander oil, almond oil, wheat germ oil, bone oil, tallow, lard, poultry fat, fish oil, and combinations thereof.

8. A process for preparing an unsaturated alcohol composition which comprises:
(a) reacting a metathesis-derived hydrocarbyl carbonyl compound in the presence of a silane compound, an organic solvent, and a catalyst system prepared from: (i) a metallic complex and (ii) a reducing agent;
(b) hydrolyzing the mixture from step (a) with a metallic base, and then adding an organic solvent; and
(c) separating, washing, drying, and/or purifying, as individual steps or in combinations thereof, the mixture of step (b) to produce the unsaturated alcohol composition.

9. The process of claim 8 wherein the metathesis-derived hydrocarbyl carbonyl compound is a metathesis-derived hydrocarbyl unsaturated ester.

10. The process of claim 8 wherein the silane compound is selected from the group consisting of alkyltrihydrosilanes, aryltrihydrosilanes, dialkyldihydrosilanes, diaryldihydrosilanes, trialkylhydrosilanes, triarylhydrosilanes, alkylhydrosiloxanes, arylhydrosiloxanes, polyalkylhydrosiloxanes, and combinations thereof.

11. The process of claim 8 wherein the metallic complex comprises the formula MXₙ, wherein M is a transition metal selected from the group consisting of zinc, cadmium, manganese, cobalt, iron, copper, nickel, ruthenium, and palladium; X is an anion such as a halide, a carboxylate or any anionic ligand; and n is a number from 1 to 4.

12. The process of claim 11, wherein the anion is selected from the group consisting of chloride, bromide, iodide, carbonate, isocyanate, cyanide, phosphate, acetate, propionate, 2-ethylhexanoate, stearate, naphthenate, zinc, cadmium, manganese, cobalt, iron, copper, nickel, ruthenium, palladium, and combinations thereof.

13. The process of claim 8, wherein the reducing agent is selected from the group consisting of lithium hydride, sodium hydride, potassium hydride, an alkaline earth metal hydride, a boron hydride, a metallic borohydride, an alkylborane, an alkoxyborane, an aluminum hydride, an organic magnesium compound, an organic lithium compound, and combinations thereof.

14. The process of claim 8, wherein the hydrolyzing of the mixture from step (a) is performed with the metallic base is selected from the group consisting of sodium hydroxide, potassium hydroxide, calcium oxide, sodium carbonate, and combinations thereof.

15. The process of claim 8, wherein the organic solvent is selected from the group consisting of an ether, aliphatic hydrocarbon, aromatic hydrocarbon, and combinations thereof.

16. The process of claim 9, wherein the metathesis-derived hydrocarbyl unsaturated ester comprises methyl-9-decenoate.

17. The process of claim 9, wherein the metathesis-derived hydrocarbyl unsaturated ester comprises methyl-9-dodecenoate.

18. The process of claim 8, wherein the unsaturated alcohol composition comprises 9-decen-1-ol.

19. The process of claim 8, wherein the unsaturated alcohol composition comprises 9-dodecen-1-ol.

20. The process of claim 8, wherein:
(a) the metathesis-derived hydrocarbyl carbonyl compound is a metathesis-derived hydrocarbyl unsaturated ester,
(b) the silane compound is selected from the group consisting of alkyltrihydrosilanes, aryltrihydrosilanes, dialkyldihydrosilanes, diaryldihydrosilanes, trialkylhydrosilanes, triarylhydrosilanes, alkylhydrosiloxanes, arylhydrosiloxanes, polyalkylhydrosiloxanes, and combinations thereof;
(c) the organic solvent is selected from the group consisting of ethers, aliphatic hydrocarbons, and aromatic hydrocarbons;
(d) the metallic complex comprises the formula MXₙ, wherein M represents a transition metal selected from the group consisting of zinc, cadmium, manganese, cobalt, iron, copper, nickel, ruthenium, and palladium, X is an anion selected from the group consisting of chloride, bromide, iodide, carbonate, isocyanate, cyanide, phosphate, acetate, propionate, 2-ethylhexanoate, stearate, naphthenate, zinc, cadmium, manganese, cobalt, iron, copper, nickel, ruthenium, palladium, and combinations thereof, and n is a number from 1 to 4;
(e) the reducing agent is selected from the group consisting of lithium hydride, sodium hydride, potassium hydride, an alkaline earth metal hydride, a boron hydride, a metallic borohydride, an alkylborane, an alkoxyborane, an aluminum hydride, an organic magnesium compound, and an organic lithium compound;
(f) the metallic base is selected from the group consisting of sodium hydroxide, potassium hydroxide, calcium oxide, sodium carbonate, and combinations thereof; and
(g) the organic solvent is selected from the group consisting of ethers, aliphatic hydrocarbons, aromatic hydrocarbons, and combinations thereof.

21. The process of claim 8, wherein:
(a) the metathesis-derived hydrocarbyl carbonyl compound is a metathesis-derived methyl-9-decenoate;
(b) the silane compound is polymethylhydrosiloxane;
(c) the organic solvent is diisopropyl ether;
(d) the metallic complex comprises the formula MXₙ, wherein M represents zinc, X represents 2-ethylhexanoate, and n is 2;
(e) the reducing agent is sodium borohydride;
(f) the metallic base is potassium hydroxide;
(g) the organic solvent is diisopropyl ether; and
(h) the unsaturated alcohol composition formed is 9-decen-1-ol.

22. The process of claim 8, wherein: preparing 9-dodecen-1-ol which comprises:
(a) the metathesis-derived hydrocarbyl carbonyl compound is a metathesis-derived methyl-9-dodecenoate;
(b) the silane compound is polymethylhydrosiloxane;
(c) the organic solvent is diisopropyl ether;
(d) the metallic complex comprises the formula MXₙ, wherein M represents zinc, X represents 2-ethylhexanoate, and n is 2;
(e) the reducing agent is sodium borohydride;
(f) the metallic base is potassium hydroxide;
(g) the organic solvent is diisopropyl ether; and
(h) the unsaturated alcohol composition formed is 9-dodecen-1-ol.

23. A derivative made by polymerization of a metathesis-derived unsaturated alcohol composition with an individual or mixed alpha olefin stream.

24. A synthetic base stock comprising the derivative of claim 23.

25. The derivative of claim 23, wherein the polymerization is carried out in the presence of an alcohol promoter and a polymerization catalyst selected from the group consisting of cationic, free radical, anionic, Ziegler-Natta, organometallic, metallocene, or ring-opening metathesis polymerization (ROMP) catalysts.

26. The derivative of claim 23, wherein the alcohol promoter comprises 9-decen-1-ol or 9-dodecen-1-ol.

27. A derivative made by reacting a metathesis-derived unsaturated alcohol composition with one of the following: (a) a sulfurizing reagent, (b) a carboxylic acid, or (c) an amine compound.

28. A saccharide derivative made by acid-catalyzed direct glycosidation of a metathesis-derived unsaturated alcohol composition, or by transglycosidation of lower polyglucosides with a metathesis-derived unsaturated alcohol composition.

29. An alkyl glyceryl ether derivative made by the alkylation of a metathesis-derived unsaturated alcohol composition with glycidol, in the presence of an acidic or alkaline catalyst.

30. A phosphoric acid mono-, di-, and tri-ester derivative made by the phosphorylation of a metathesis-derived unsaturated alcohol composition using phosphorus pentoxide.
